(19) United States
(12) Patent Application Publication
(10) Pub. No.: US 2023/0176074 A1
(43) Pub. Date: Jun. 8, 2023

(54) BIOMARKERS FOR THE DIAGNOSIS OF RESPIRATORY TRACT INFECTIONS

(71) Applicant: B.R.A.H.M.S GmbH, Hennigsdorf (DE)

(72) Inventors: Michael Hausmann, Nyon (CH); Ingo Curdt, Berlin (DE); Stefan Gehrig, Berlin (DE); Christiane Dinter, Berlin (DE)

(73) Assignee: B.R.A.H.M.S GmbH, Hennigsdorf (DE)

(21) Appl. No.: 17/917,907

(22) PCT Filed: Apr. 9, 2021
(86) PCT No.: PCT/EP2021/059264

§ 371 Date: Oct. 7, 2022

(30) Foreign Application Priority Data
Apr. 9, 2020 (EP) 20169058.3
Apr. 9, 2020 (EP) 20169071.6

(51) Int. Cl. G01N 33/68

(52) U.S. Cl. CPC G01N 33/6893; G01N 2333/4703; G01N 2333/4745; G01N 2800/127

(57) ABSTRACT

The present invention relates to a method for diagnosing a respiratory tract infection in a subject, comprising determining in a sample from said subject the level of High-Mobility-Group-Protein B1 (HMGB1), and/or determining in a sample from said subject the level of a histone protein, and/or determining in a sample from said subject the level of Insulin-like growth factor binding protein, acid labile subunit (IGFALS), wherein the subject is diagnosed with a bacterial respiratory tract infection when the level of IGFALS is below a predetermined threshold level and/or the level of the histone protein is above a predetermined threshold value, and/or wherein the subject is diagnosed with a respiratory tract infection when the level of HMGB1 is above a predetermined threshold level.

### FIELD OF THE INVENTION

The present invention relates to markers and methods for the diagnosis and differential diagnosis of respiratory tract infections (RTI) and in particular lower respiratory tract infections (LRTI) like pneumonia.

### BACKGROUND OF THE INVENTION

A serious problem in clinical settings is the diagnosis of patients with disorders having overlapping symptoms or unspecific disease-related characteristics. In addition, fast patient management and correct treatment initiation despite increasingly overloaded clinical settings like the emergency department (ED) or primary care is desired.

There exists a clear problem in the triage of patients with symptoms of a Lower Respiratory Tract Infection (LRTI) such as shortness of breath, weakness, coughing, fatigue, confusion, pleuritic chest pain or fever, because the shown symptoms can vary from patient to patient, depend from the severity of the disease and are overlapping with non-infection-caused diseases or disorders such as chronic obstructive pulmonary disease (COPD), acute coronary syndrome (ACS), asthma, heart failure, lung embolism, tumor, especially tumor of the lung, oedema, idiopathic pneumonia syndrome (IPS) or atrial fibrillation (Girish et al., Med Clin N Am 95 (2011) 1143-1161). Many of the underlying diseases can quickly shift the patient in a severe, life threatening condition like sepsis and gives no room for time-consuming causative diagnostic procedures or scoring systems with an overall time of hours or even days. This is a serious problem for the medical staff and there is a strong medical need for a fast, personalized treatment and medical decision of such patients.

LRTI such as pneumonia, acute bronchitis and bronchiolitis are caused by pathogenic, infectious agents and needs a pathogen specific antimicrobial or antiparasitic treatment as well as a patient protective isolation to avoid superinfections or further spreads.

Among Lower Respiratory Tract Infections, pneumonia is a serious health problem and one of the major causes of mortality and morbidity worldwide. It is caused by a broad spectrum of bacterial, viral and, in rare cases, fungal pathogens or other parasites (Raeven et al., BMC Infectious Diseases (2016) 16:299; Li et al., Microbes and Infection (2020) 22(2):80-85). The economic burden is high and cost drivers are hospitalization and length of stay of the patient.

The most prevalent form of pneumonia is the community acquired pneumonia (CAP) which is an important cause of death, mostly for children below 5 years of age, in adults above 65 or in patients with comorbidities. The incidence of CAP is expected to increase in the next decade due to the ageing population and the subsequent increase in comorbidities. Therefore, major risks for CAP can be primary morbidities such as chronic disorders like asthma or COPD, chronic heart failure, an incompetent immune system or the use of drugs like proton pump inhibitors as well as respiratory stress factors like air pollution or smoking. The mortality rates ranging from <1% up to 50%, depending on the healthcare system and patient setting (Girish et al., loc. cit.; Cillioniz et al., Int. J. Mol. Sci. (2016) 17:2120; Savvateeva et al., BioMed Res Int (2019) 1701276).

Pneumonia can be caused by a multiplicity of pathogens, which can cause slightly different symptoms and their spread can vary by region and season (Ho et al., Infect Dis Clin N Am (2019) 33: 1087-1103). A key factor for the decrease in mortality of the patients are the fast and effective, pathogen related treatment with appropriate antimicrobial drugs and other supportive medicinal applications like oxygenation or mechanical ventilation.

The sickening bacteria can be divided into a typical and atypical group.

The most common pathogens causing CAP are extracellular bacteria including Streptococcus pneumoniae, Hemophilus influenzae, Moraxella catarrhalis, Staphylococcus aureus, especially methicillin-resistant Staphylococcus aureus (MRSA).

The atypical group, as second most frequent class of CAP pathogens, is amplifying intracellularly in human cells and usually has no typical bacterial cell wall. Atypical representatives are Legionella pneumophila, Mycoplasma pneumoniae, Chlamydophila pneumoniae, Chlamydophila psittaci and Coxiella burnetii. The proportion of atypical pneumonia is often reported between 5-30% of cases with 30% mixed infections and seems to be more common in patients admitted to the ICU (up to 20%). Patients infected with atypical bacteria often show subacute symptoms like non-productive cough, low fever, normal WBCs and frequently associated extra pulmonary manifestations.

CAP-infection rates that are caused by respiratory viruses vary from 2% to 30% of the cases. Some studies found a higher frequency of viral caused CAP than previously thought, beside pandemic or epidemic events with an enormous increase of cases. The most common viral particles are the influenza virus, respiratory syncytial virus (RSV), coronavirus, rhinovirus, parainfluenza viruses, human metapneumovirus, varicella, hantavirus and adenovirus. In viral infections, one of the most problematic complication are coinfections with other pathogens that can frequently shift the patient’s situation towards a more severe state. The interplay between the virus particles and bacteria are not fully understood but is likely that the interaction increases the bacterial virulence resulting in poor clinical outcomes (Girish et al., loc. cit.; Cillioniz et al., loc. cit.; Savvateeva et al., loc. cit.) as well as an increased burden of the immune system, because of the high load of pathogenic particles.

The identification of the underlying pathogen or at least ruling out of certain pathogens is the key for an accurate diagnosis, treatment decision and the containment of the infection in a population.

An accurate diagnosis of pneumonia with the underlying pathogenic cause cannot be made based on the assessment of patient’s symptoms and signs alone. Additional clinical examinations or imaging support the diagnosis of a pneumonia but cannot distinguish between different pathogens. Imaging methods like x-ray, sonography or CT-scan have disadvantages, because of their limited availability, the interobserver variability in the interpretation of results and difference in the experience of the radiologists or sonographer.

No standard serological test for all patients with RTI and in particular CAP exists, let alone a fast and easy clinical tool that would make it possible to distinguish intracellular pathogens from classical pathogens.

Direct microbiological identification of pathogens from sputum, bronchoalveolar lavage or blood depends strongly on the quality of the obtained sample material and takes hours to several days.

The current IDSA/ATS guideline 2019 recommends a sputum or blood culture only in patients with a suspicion of a resistant pathogen, no routine determination of biomarkers or urinary antigen testing and no follow up x-rays, but an empirical administration of antibiotics (Metlay et al., Am J Respir Crit Care Med (2019) 200(7): e45-e67).

The before mentioned culturing has a rate of pathogen identification in CAP of below 50%. One reason is the difficulty to grow atypical bacteria in standard culture media, because they are located intracellularly and/or have no typical cell wall and therefore are not easy to identify.

Another method is the identification by molecular biological techniques like Polymerase Chain Reaction (PCR) that are commonly used for the identification of viral caused infections. Here it is important to know, that the upper respiratory tract in healthy people are often colonized by potentially pathologic bacteria like Pseudomonas aeruginosa. If the samples material was not taken in the lower respiratory tract or includes colonized, “harmless” microbes from the upper respiratory tract, the systems tend to show false positive results that would end in unnecessary treatment (Savvateeva et al, loc. cit.). In contrast to that the host response biomarkers detected in the here described approach are altered only if the patient immune system reacts to an infection and therefore prevents false positives.

There are current strategies with blood host biomarkers, but the clinical use of a single biomarker is limited. Currently, there are no biomarker-based algorithms for establishing the aetiology of CAP. No known single biomarker can reliably differentiate different pathogens to make clinical decisions regarding treatment. Procalcitonin for example is a well-known biomarker for the diagnosis and severity of a bacterial infection. Other published biomarkers like TRAIL (WO 2016/059636 A1) or MxA (WO 2014/137858 A1) have been used for the detection of a viral cause. Nevertheless, the feasibility of known single biomarkers is limited for the identification or differentiation of a viral vs. bacterial and especially the sub-differentiation between typical and atypical bacterial infections (Kruger et al., Respiratory Research (2009) 10:65).

Complex severity assessment scores such as Pneumonia Severity Index (PSI), the CURB-65 criteria (a modification of the British Thoracic Society scoring system) or the SMART-COP have been used to stratify patients according to the risk of mortality and are used for the guidance of medical care, but are time-consuming and difficult to calculate.

Often, the identity of the causative pathogen remains unknown and therefore the treatment of patients frequently follows a time-consuming trial and error method.

In the current practice antibiotics are the first line treatment for pneumonia. However, they are not effective or indicated for parasitic or viral infections, but nevertheless about 41% of the overall antibiotic use are in connection with respiratory conditions (Ho et al., loc. cit.).

Medical guidelines recommend an empiric procedure, because of the need of a therapeutic intervention within the first hours after patient admission to the ED or within the limited timeslot in the general practitioner setting (Thibodeau et al., American Family Physician (2004) 69(7): 1699-1706).

This prescription practice causes a far higher antibiotic consumption and overtreatment with broad-spectrum antibiotics, which can create additional costs and drug-side effects. It has been shown that the use of antibiotics in non-infected morbid patients with symptoms that mimics respiratory tract infection such as patients with acute heart failure and dyspnea increases the risk of side effects and bad outcome (Girish et al., loc. cit.; Maisel et al., Eur J Heart Fail. (2012) 14:278-286).

Another consequence is the development of multi-resistant bacteria that significantly limit the usability of new antibiotics.

The increasing antibiotic resistance of S. pneumoniae, the most common cause of CAP, to several antibiotics such as cephalosporins, macrolides and fluoroquinolones can become a worldwide issue. Within the last two decades between 20% and 30% of pneumococcus disease cases worldwide are resistant to more than three classes of antibiotics.

Based on the intracellular appearance and non-existing cell wall, the treatment against atypical pneumonia pathogens requires different types of antibiotics compared to bacterial pathogens in typical pneumonia. An antibiotic for atypical bacteria must be able to enter human cells and must not be directed against typical bacterial cell wall types. Therefore, the standard beta-lactams are not effective and other antimicrobials such as erythromycin and sometimes tetracycline have been traditionally used in atypical infections. Macrolide antibiotics are better tolerated than erythromycin. Doxycycline has fewer gastrointestinal side effects and is a less expensive alternative. Fluoroquinolones have an excellent bioavailability that allows for a once daily dosing (Thibodeau et al., loc. cit.).

Effective patient management helps decreasing costs but requires fast and right decisions about the clinical setting (outpatient, admission to the hospital or intensive care unit (ICU)) and the selection of appropriate treatment.

One solution of the current problems is the use of advanced technologies with rapid diagnostics that enable an etiologic agent-targeted therapy (Ho et al., loc. cit.).

One of the most important clinical needs is the early identification of the most causative pathogen groups, highlighting the differentiation between bacterial and viral pathogens in RTI and also an early and fast sub-differentiation of atypical and typical bacteria for an effective management and accurate treatment of patients and the avoidance of the currently used empirical “trial and error” method.

Therefore, a fast and reliable detection of host biomarkers, and in particular blood biomarkers, is the solution for the identification and differential diagnosis of the causative pathogen of an infection-based disorder and/or the rule-out of non-infectious disorders with overlapping or mimicking symptoms. Rapid tests or automated assays are easy to handle and can support the decision of the patients triage and the initial, accurate and personalized treatment.

A clear medical advantage of those procedure would be a decrease of antibiotic consumption in general or at least the avoidance of unnecessary application thereof that would also decrease the generation of further antibiotic resistances. Another advantage is the possibility of fast medical decision making and the initiation of the right medicinal interventions. Another group of clinically relevant pathogens are viruses that tend to cause pneumonia. In the case of a critical situation like a epedemia or pandemia, like in the case of so far unknown pathogenic variants like the coronavirus related Severe Acute Respiratory Syndrome-CoV (SARS-CoV) 2002, Middle East Respiratory Syndrome-CoV (MERS-CoV) 2012 or SARS-CoV 2/ COVID-19 (M. Ashour et al. Pathogens 2020, 9(3)), the medical system has a high amount of patients with a potential risk of being infected with the new pathogen, but can also be suffer from non-infection related critical disorders with similar symptoms as well as co-infections with bacteria. In that case, the fast triage of patients is the most important tool for the decision of the next clinical steps and the provision of the right medical support to decrease the risk of complications such as dyspnea, ARDS, sepsis, (septic) shock or mortality of the patient as good as possible.

### SUMMARY OF THE INVENTION

The present invention relates to a method for diagnosing a respiratory tract infection in a subject, comprising
determining in a sample from said subject the level of High-Mobility-Group-Protein B1 (HMGB1), and/or
determining in a sample from said subject the level of a histone protein, preferably selected from histone H4, histone H2A, histone H2B, histone H3 and histone H1, and/or
determining in a sample from said subject the level of Insulin-like growth factor binding protein, acid labile subunit (IGFALS),
wherein the subject is diagnosed with a respiratory tract infection when the level of HMGB1 is above a predetermined threshold level, and/or
wherein the subject is diagnosed with a bacterial respiratory tract infection when the level of IGFALS is below a predetermined threshold level and/or the level of the histone protein is above a predetermined threshold value.

In other words, the present invention in one aspect relates to a method for diagnosing a respiratory tract infection in a subject, comprising
determining in a sample from said subject,
   (i) the level of High-Mobility-Group-Protein B1(HMGB 1),
   (ii) the level of a histone protein, and/or
   (iii) the level of Insulin-like growth factor binding protein, acid labile subunit (IGFALS), wherein the subject is diagnosed with a respiratory tract infection when the level of HMGB 1 is above a predetermined threshold level, and/or
wherein the subject is diagnosed with a bacterial respiratory tract infection when the level of IGFALS is below a predetermined threshold level, and/or the level of the histone protein is above a predetermined threshold value.

The present invention relates in one aspect to a method for diagnosing a respiratory tract infection in a subject suspected of having a respiratory tract infection (RTI), comprising determining in a sample from said subject the level of High-Mobility-Group-Protein B1 (HMGB 1), wherein the subject is diagnosed with a respiratory tract infection when the level of HMGB 1 is above a predetermined threshold level.

In another aspect, the present invention relates to a method for the differential diagnosis of a disease of the respiratory tract in a subject, comprising
determining in a sample from said subject the level of High-Mobility-Group-Protein B1 (HMGB1),
wherein the subject is diagnosed with a respiratory tract infection (RTI) when the level of HMGB1 is above a predetermined threshold level.

In this context, the subject may have one or more symptoms of a lower respiratory tract infection (LRTI), e.g. said subject may show one or more symptoms selected from shortness of breath, weakness, fever, sputum formation, coughing, fatigue, wheezing, chest discomfort or pain, rapid breathing, difficulty breathing, congestion, runny nose and sore throat. Said subject may also suffer from cough and one or more symptoms selected from sputum formation, shortness of breath, wheezing and chest discomfort or pain. The method is particularly useful to differentiate subjects with symptoms that overlap between RTI and other diseases of the lower respiratory tract (“RTI mimics”).

In the context of the present invention, the RTI can especially be a lower respiratory tract infection (LRTI), e.g. selected from the group of acute bronchitis, pneumonia and bronchiolitis. The LRTI can be a bacterial or a viral infection such as an atypical bacterial infection, particularly an atypical bacterial pneumonia. The methods of the invention are particularly useful for distinguishing subjects with an atypical bacterial LRTI such as an atypical bacterial pneumonia from healthy subjects or from subjects having symptoms mimicking that of an LRTI, e.g. COPD patients, see below.

Herein, the viral infection may for instance be selected from the group consisting of Influenza A, Influenza B, Severe acute respiratory syndrome (SARS), Middle East respiratory syndrome (MERS) and Coronavirus disease 2019 (COVID-19).

In the context of the present invention, additionally the level of one or more further markers selected from the group consisting of procalcitonin (PCT), proadrenomedullin (proADM) or a fragment thereof, histone protein, Serum amyloid A1 (SAA1), Fetuin-A (FetA), Insulin-like growth factor binding protein, acid labile subunit (IGFALS), Tumor Necrosis Factor Related Apoptosis Inducing Ligand (TRAIL) and C-X-C motif chemokine 10 (CXCL10) can be determined in a sample from said subject in order to improve the diagnosis. In a particular case, the level of MR-proADM is determined in a sample from said subject; in particular wherein the level of MR-proADM is indicative for the severity of the infection.

Also, additionally to HMGB1 the level of one or histone proteins selected from histone H2B, histone H4, histone H2A, histone H3 and histone H1 can be determined in a sample from said subject, preferably the level of H4 is determined. The subject may be diagnosed with atypical bacterial pneumonia when the level of the at least one histone protein, preferably H4, is above a predetermined threshold level. On the other hand, the subject may be diagnosed with typical bacterial pneumonia when the level of the at least one histone protein, preferably H4, is above a first predetermined threshold level and below a second predetermined threshold level.

Moreover, additionally the level of FetA may be determined in a sample from said subject. The subject may be diagnosed with a bacterial LRTI when the level of FetA is below a predetermined threshold level. On the other hand, the subject may be diagnosed with a viral LRTI when the level of FetA is above a predetermined threshold level.

Furthermore, additionally to HMGB1 the level of IGFALS may be determined in a sample from said subject. The subject may be diagnosed with a bacterial LRTI when the level of IGFALS is below a predetermined threshold level. On the other hand, the subject may be diagnosed with a viral LRTI when the level of IGFALS is above a predetermined threshold level.

Also, additionally the level of CXCL10 may be determined in a sample from said subject. The subject may be diagnosed with a viral LRTI when the level of CXCL10 is above a predetermined threshold level.

Further, additionally the level of TRAIL may be determined in a sample from said subject. The subject may be diagnosed with a viral LRTI when the level of TRAIL is above a predetermined threshold level.

The present invention in a further aspect relates to a method for diagnosing a respiratory tract infection in a subject, comprising
determining in a sample from said subject the level of a histone protein, preferably selected from histone H4, histone H2A, histone H2B, histone H3 and histone H1, and/or determining in a sample from said subject the level of Insulin-like growth factor binding protein, acid labile subunit (IGFALS),
wherein the subject is diagnosed with a bacterial respiratory tract infection when the level of IGFALS is below a predetermined threshold level and/or the level of the histone protein is above a predetermined threshold value. Particularly, the histone protein may be H4.

A typical subject herein is a subjected that is suspected of having a bacterial respiratory tract infection. Said subject may have one or more symptoms of a lower respiratory tract infection (LRTI), particularly pneumonia. Said subject may show one or more symptoms selected from shortness of breath, weakness, fever, sputum formation, coughing, fatigue, wheezing, chest discomfort or pain, rapid breathing, difficulty breathing, congestion, runny nose and sore throat. Particularly, said subject may suffer suffer from cough and one or more symptoms selected from sputum formation, shortness of breath, wheezing and chest discomfort or pain.

In this context, additionally the level of one or more further markers selected from the group consisting of procalcitonin (PCT), proadrenomedullin (proADM) or a fragment thereof, histone protein, High-Mobility-Group-Protein B1 (HMGB1), Serum amyloid A1 (SAA1), Fetuin-A (FetA), Tumor Necrosis Factor Related Apoptosis Inducing Ligand (TRAIL) and C-X-C motif chemokine 10 (CXCL10) may be determined in a sample from said subject. For instance, the level of MR-proADM may be determined in a sample from said subject; in particular, wherein the level of MR-proADM is indicative for the severity of the infection.

Moreover, the level of HMGB1 may be determined additionally to the level of the histone protein or the level of IGFALS in a blood sample from said subject, wherein the subject is diagnosed with a bacterial respiratory tract infection when the level of IGFALS is below a predetermined threshold level, the level of the histone protein is above a predetermined threshold value and the level of HMGB1 is above a predetermined threshold level.

Herein, the subject is diagnosed with atypical bacterial pneumonia when the level of the at least one histone protein, preferably H4, is above a predetermined threshold level. On the other hand, the subject may be diagnosed with typical bacterial pneumonia when the level of the at least one histone protein, preferably H4, is above a first predetermined threshold level and below a second predetermined threshold level.

In the context of the present invention, the sample is a sample of a bodily fluid, preferably a blood sample, a saliva sample, nasal swab, sweat sample, a urine sample or a bronchoalveolar lavage (BAL), more preferably serum, plasma or whole blood, most preferably plasma.

The invention also relates to an antibiotic for use in the treatment of a bacterial respiratory tract infection in a subject, wherein said subject is treated with the antibiotic if it has been determined to have a bacterial respiratory tract infection with a method according to the invention.

### BRIEF DESCRIPTION OF THE DRAWINGS

FIG. 1: shows the concentrations of HMGBI (in ng/ml) in blood samples from healthy donors and patients with typical bacterial pneumonia (typ.), atypical bacterial pneumonia (atyp.) and viral pneumonia as determined by ELISA.

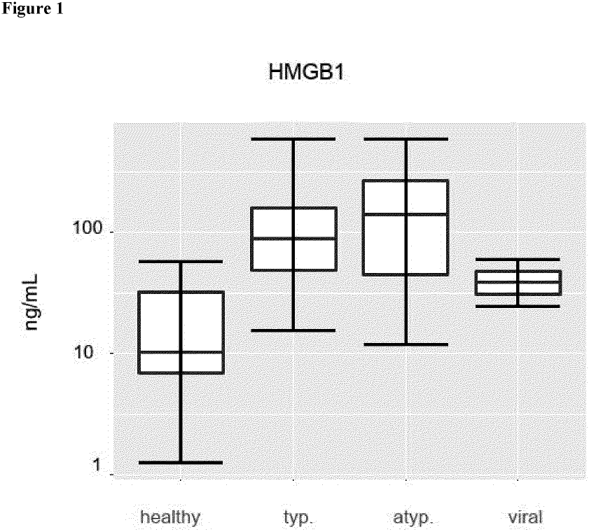

FIG. 2: shows the relative concentrations of histone H4 in blood samples from healthy donors and patients with typical bacterial pneumonia (typ.), atypical bacterial pneumonia (atyp.) and viral pneumonia as determined by MS.

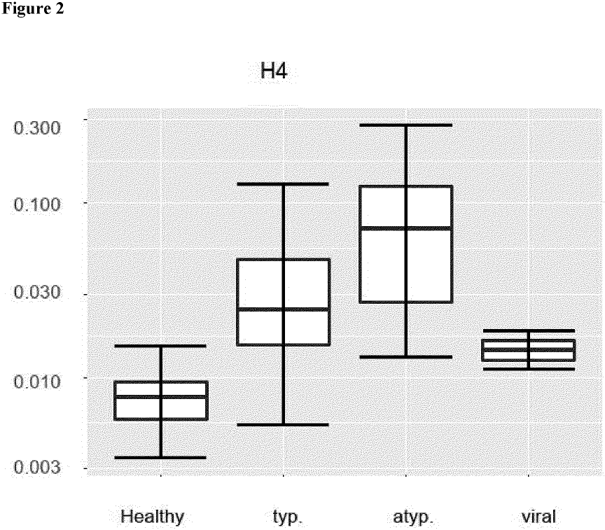

FIG. 3: shows the relative concentrations of IGFALS in blood samples from healthy donors and patients with bacterial respiratory tract infections (11 pneumonia, 7 non-pneumonia), viral respiratory tract infections (4 pneumonia, 26 non-pneumonia) and atypical bacterial pneumonia as determined by ELISA.

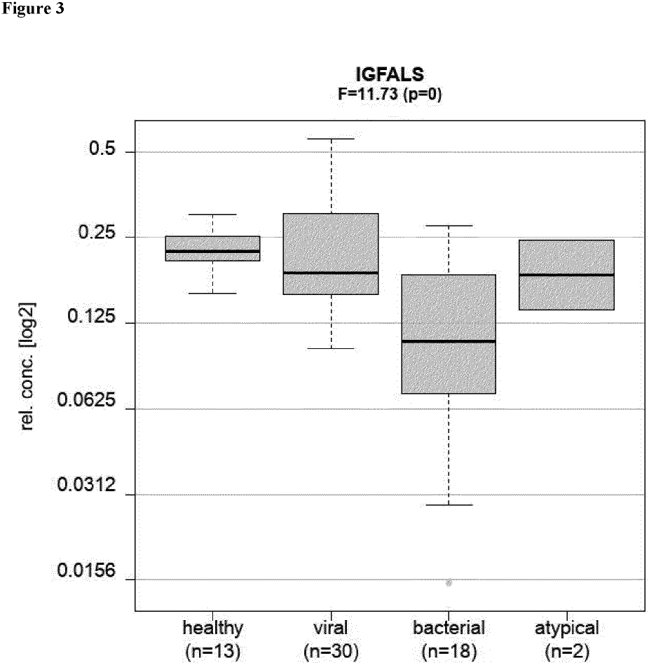

FIG. 4: shows the relative concentrations of Fetuin-A in blood samples from healthy donors and patients with bacterial respiratory tract infections (11 pneumonia, 7 non-pneumonia), viral respiratory tract infections (4 pneumonia, 26 non-pneumonia) and atypical bacterial pneumonia as determined by a magnetic bead-based multiplex immunoassay.

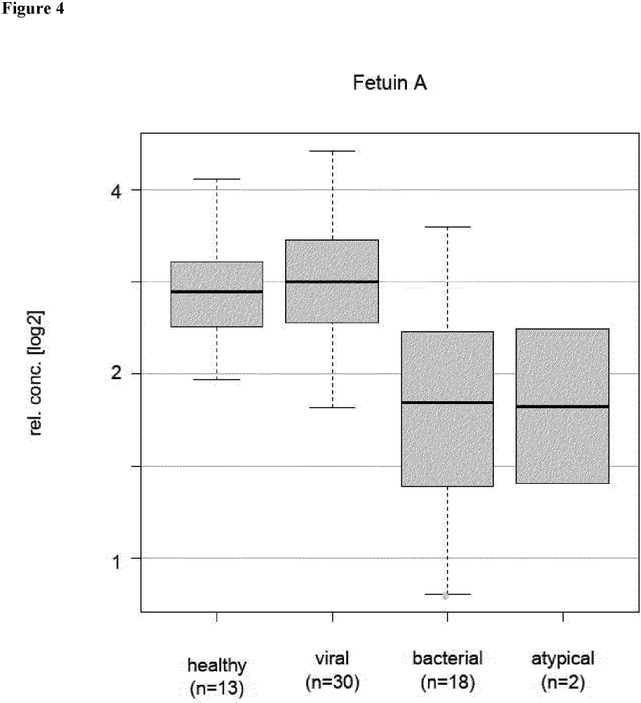

### DETAILED DESCRIPTION OF THE INVENTION

An object of the invention is to provide markers and methods for the diagnosis and differential diagnosis of respiratory tract infections (RTIs) and in particular lower respiratory tract infections (LRTIs). In particular, the acute forms of RTI/LRTI are diagnosed with the methods of the present invention. As outlined herein above, the present invention is based on the surprising finding that the following markers can be used in the differential diagnosis of respiratory tract infections and in particular lower respiratory tract infections: High-Mobility-Group-Protein B1(HMGB1), histone proteins such as histone H4, histone H2A, histone H2B, histone H3 and histone H1, Insulin-like growth factor binding protein, acid labile subunit (IGFALS) and Fetuin-A (FetA). As is evident from the enclosed Example and Figures, these markers can contribute to this diagnosis and in particular to the differentiation of RTI from diseases with similar or identical symptoms or in differentiating bacterial from viral RTI, in particular LRTI, more in particular pneumonia, or in differentiating typical from atypical bacterial pneumonia.

Herein, the methods are preferably used for the diagnosis of LRTIs. Hence, the RTI is in all aspects and embodiments (unless stated otherwise) preferably an LRTI. Pneumonia is one of the most severe forms of LRTI. Hence, it is in all aspects and embodiments of the present invention intended to diagnose pneumonia in particular. The RTIs/LRTIs/pneumonia can in the context of the present invention be of different origin such as caused by bacterial or viral pathogens. In the case of bacterial pneumonia, there are typical and atypical forms of pneumonia caused by different bacteria; see discussion below.

A respiratory tract infection is an infectious disease involving the respiratory tract. An RTI can be further classified as an upper respiratory tract infection (URTI) or a lower respiratory tract infection (LRTI).

The upper respiratory tract is generally considered to be the airway above the glottis or vocal cords. This includes the nose, sinuses, pharynx, and larynx. Typical infections of the upper respiratory tract include tonsillitis, pharyngitis, laryngitis, sinusitis, otitis media, certain types of influenza, and the common cold. Symptoms of URTIs can include cough, sore throat, runny nose, nasal congestion, headache, low grade fever, facial pressure and sneezing.

The lower respiratory tract consists of the trachea (windpipe), bronchial tubes, the bronchioles, and the lungs. Lower respiratory tract infections such as pneumonia are generally more serious than upper respiratory tract infections. LRTIs are the leading cause of death among all infectious diseases. The two most common LRTIs are bronchitis and pneumonia. Another LRTI is bronchiolitis. Influenza or coronavirus affects both the upper and lower respiratory tracts. Symptoms of LRTIs in general include shortness of breath, weakness, fever, coughing and fatigue up to Acute Respiratory Distress Syndrome (ARDS), organ dysfunction and sepsis.

Bronchitis is inflammation of the bronchi (large and medium-sized airways) in the lungs that causes coughing. Symptoms include coughing up sputum, wheezing, shortness of breath, and chest pain. Bronchitis can be acute or chronic. Acute bronchitis usually has a cough that lasts around three weeks. In more than 90% of cases the cause is a viral infection. These viruses may be spread through the air when people cough or by direct contact. Typically, these viral infections are rhinovirus, parainfluenza, coronavirus or influenza. A small number of cases are caused by a bacterial infection such as Mycoplasma pneumoniae or Bordetella pertussis.

Pneumonia is an inflammatory condition of the lung affecting primarily the small air sacs known as alveoli and can be community acquired (CAP) or hospital acquired (HAP) (nosocomial). Typically, symptoms include some combination of productive or dry cough, chest pain, fever and difficulty breathing. Bacteria are the most-common cause of community-acquired pneumonia (CAP), with Streptococcus pneumoniae isolated in nearly 50% of cases. Other commonly isolated bacteria include Haemophilus influenzae, Chlamydophila pneumoniae, Mycoplasma pneumoniae, Staphylococcus aureus, Moraxella catarrhalis, Legionella pneumophila and Gram-negative bacilli. A number of drug-resistant versions of the above infections are becoming more common, including drug-resistant Streptococcus pneumoniae (DRSP) and methicillin-resistant Staphylococcus aureus (MRSA).

In adults, viruses account for approximately a third and in children for about 15% of pneumonia cases. Common causes of viral pneumonia are: Influenza virus A and B, Respiratory syncytial virus (RSV) and human parainfluenza viruses. Further viruses that commonly result in pneumonia include: Adenoviruses, Metapneumovirus, Hantaviruses, Coronavirus in particular special forms, mutations/variants, or zoonotic related viruses such as Severe acute respiratory syndrome Coronavirus(SARS CoV), Middle East respiratory syndrome Coronavirus(MERS CoV), Severe acute respiratory syndrome Coronavirus 2 (SARS-CoV-2/ COVID-19). The SARS coronavirus causes severe acute respiratory syndrome (SARS). The MERS coronavirus causes Middle East respiratory syndrome (MERS). SARS-CoV-2 causes Coronavirus disease 2019 (COVID-19).

Bronchiolitis is blockage of the small airways in the lungs due to a viral infection which usually only occurs in children less than two years of age. Symptoms may include fever, cough, running nose, wheezing, and breathing problems.

HMGB1 is especially useful for diagnosing an RTI (bacterial and viral), particularly LRTI, in a subject, and in distinguishing an RTI, particularly LRTI, from healthy individuals or from patients with diseases that have similar or overlapping symptoms, i.e. that mimic RTIs and LRTIs.

Similarly, histone proteins (including histone H4, histone H2A, histone H2B, histone H3 and histone H1) are useful markers for diagnosing an RTI (bacterial and viral), particularly LRTI, in a subject, and in distinguishing an RTI, particularly LRTI, from healthy individuals or from patients with diseases that have similar or overlapping symptoms, i.e. that mimic RTIs and LRTIs. Histones are also useful for the differentiation between atypical and typical bacterial pneumonia.

In contrast, Fetuin A (FetA) is particularly useful in distinguishing bacterial from viral RTI/LRTI and IGFALS is particularly useful in distinguishing typical from atypical bacterial pneumonia as well as viral from typical bacterial infection.

Hence, in one aspect of the present invention, it is the aim to differentiate subjects with RTI/LRTI from subjects having a disease with similar or overlapping symptoms like acute coronary syndrome (ACS), Asthma, Chronic obstructive pulmonary disease (COPD), Heart Failure, Lung Embolism, Tumor of the lung, other Tumors or Atrial Fibrillation.

Given their individual strengths, the markers of the invention can be combined (as panels of at least 2, 3 or 4 markers) in order to obtain a more detailed differential diagnosis and/or to direct or monitor adequate therapy. In addition, the above markers, either individual or as panels, can be combined with further markers or other clinical parameters to further improve diagnosis. Such additional markers include procalcitonin (PCT), proadrenomedullin (proADM) and fragments thereof such as MR-proADM, mature ADM, PAMP, Serum amyloid A1 (SAA1), Interferon-induced GTP-binding protein Mx1 (Mx1), Tumor Necrosis Factor Related Apoptosis Inducing Ligand (TRAIL), C-X-C motif chemokine 10 (CXCL10; also known as IP10) and C-reactive Protein (CRP).

Proadrenomedullin (proADM) and fragments thereof, preferably, MR-proADM, can in particular be used as an additional marker for the severity of the RTI/LRTI. The higher the level of proADM, particularly MR-proADM, the more severe the disease is.

The present invention relates to several aspects which are discussed in the following.

In one aspect the present invention relates to a method for diagnosing a respiratory tract infection (RTI), particularly an LRTI, in a subject suspected of having a respiratory tract infection, comprising
determining in a sample from said subject the level of High-Mobility-Group-Protein B1 (HMGB 1),
wherein the subject is diagnosed with a respiratory tract infection when the level of HMGB1 is above a predetermined threshold level.

In a related aspect, the present invention relates to a method for the differential diagnosis of a disease of the respiratory tract in a subject, comprising
determining in a sample from said subject the level of High-Mobility-Group-Protein B1 (HMGB1),
wherein the subject is diagnosed with a respiratory tract infection, particularly LRTI, when the level of HMGB1 is above a predetermined threshold level.

In this context, HMGB1 is a particularly useful marker for distinguishing patients with atypical LRTI, particularly atypical bacterial pneumonia, from other non-bacterial related diseases mimicking pneumonia or from healthy subjects.

In another aspect, the present invention relates to a method for diagnosing a respiratory tract infection, particularly a LRTI, in a subject, comprising
determining in a sample from said subject the level of at least one histone protein, preferably selected from histone H4, histone H2A, histone H2B, histone H3 and histone H1, and/or
determining in a sample from said subject the level of Insulin-like growth factor binding protein, acid labile subunit (IGFALS),
wherein the subject is diagnosed with a bacterial respiratory tract infection when the level of IGFALS is below a predetermined threshold level and/or the level of the histone protein is above a predetermined threshold value.

In a related aspect, the present invention pertains to a method for the differential diagnosis of a bacterial pneumonia in a subject, comprising
determining in a sample from said subject the level of at least one histone protein, preferably selected from histone H4, histone H2A, histone H2B and histone H3,
wherein the subject is diagnosed with a typical bacterial pneumonia when the level of said histone protein is below a predetermined threshold level, and
wherein the subject is diagnosed with an atypical bacterial pneumonia when the level of said histone protein is above a predetermined threshold level.

In another related aspect, the present invention pertains to a method for the differential diagnosis of a respiratory tract infection in a subject, comprising
determining in a sample from said subject the level of IGFALS,
wherein the subject is diagnosed with a bacterial respiratory tract infection when the level of IGFALS is below a predetermined threshold level, and
wherein the subject is diagnosed with a viral LRTI when the level of IGFALS is above the predetermined threshold level.

Such a differential diagnosis of viral vs. bacterial RTI/LRTI/pneumonia can be improved by the combination of IGFALS with one or more markers selected from TRAIL, Mx1, FetA, CRP and CXCL10.

The combination of histone protein and IGFALS is particularly useful for the differential diagnosis of a typical bacterial LRTI, in particular a typical bacterial pneumonia.

In a similar aspect, the present invention relates to a method for diagnosing a respiratory tract infection, particularly a LRTI, in a subject, comprising
determining in a sample from said subject the level of at least one histone protein, preferably selected from histone H4, histone H2A, histone H2B and histone H3, and/or
determining in a sample from said subject the level of Fetuin-A (FetA),
wherein the subject is diagnosed with a bacterial respiratory tract infection when the level of FetA is below a predetermined threshold level and/or the level of the histone protein is above a predetermined threshold value.

The combination of histone protein and FetA is particularly useful for the differential diagnosis of a LRTI (bacterial vs. viral), in particular a bacterial pneumonia (typical and atypical).

In a related aspect, the present invention pertains to a method for the differential diagnosis of a bacterial respiratory tract infection in a subject, comprising
determining in a sample from said subject the level of Fetuin A
wherein the subject is diagnosed with a bacterial respiratory tract infection when the level of Fetuin A is below a predetermined threshold level.

The predetermined threshold values can for example be based on respective marker levels in samples from one or more individuals from a control group, e.g. healthy subjects. Depending on the differential diagnosis of interest, the control groups can be patients with a particular form of RTI (e.g. bacterial/viral infection, atypical/typical pneumonia and the like). Most preferably, the predetermined threshold is determined as a cut-off vis-à-vis the levels from the control group based on the desired specificity/sensitivity of the assay.

The present invention also relates to methods of treating a bacterial RTI, in particular a bacterial LRTI, more in particular a bacterial pneumonia, in a subject with an antibiotic, wherein the subject has been diagnosed with a bacterial RTI or bacterial LRTI or bacterial pneumonia with a method of the present invention. Correspondingly, the present invention pertains to an antibiotic for use in such a method of treating a bacterial RTI, in particular a bacterial LRTI, more in particular a bacterial pneumonia. This also includes mixed bacterial/viral infections.

Different antibiotics are commonly prescribed for typical and atypical RTI/LRT/pneumonia: Typical bacterial RTI/LRTI/pneumonia is commonly treated with amoxicillin, erythromycin, cefuroxime, flucloxacillin, doxycycline, second generation cephalosporins such as cefaclor, ciprofloxacin, or rifampicin. Atypical bacterial RTI/LRTI/pneumonia is commonly treated with macrolide antibiotics such as azithromycin and clarithromycin, fluoroquinolones such as ciprofloxacin and levofloxacin, tetracycline antibiotics such as doxycycline and tetracycline.

The most common causative bacteria of typical pneumonia are bacteria such as Streptococcus pneumoniae, Staphylococcus aureus, Haemophilus influenzae, Klebsiella pneumoniae, Escherichia coli, Pseudomonas aeruginosa and Moraxella catarrhalis. Hence, for the treatment of typical pneumonia, antibiotics directed at these bacteria are preferred.

The most common causative bacteria of atypical pneumonia are (atypical bacterial specifications such as an intracellular living cycle or without cell wall) bacteria such as Chlamydophila pneumoniae, Chlamydophila psittaci, Coxiella burnetii, Francisella tularensis, Legionella pneumophila and Mycoplasma pneumoniae. Hence, for the treatment of atypical pneumonia, antibiotics directed at bacteria with an intracellular replication cycle or without cell wall are preferred.

Hence, the present invention relates to an antibiotic for the treatment of a bacterial pneumonia in a subject,
wherein said antibiotic is selected from the group consisting of amoxicillin, erythromycin, cefuroxime, flucloxacillin, doxycycline, second generation cephalosporins such as cefaclor, ciprofloxacin, rifampicin, and
wherein said subject is treated with said antibiotic if it has been determined to have a typical bacterial pneumonia in the subject with the method according to the invention.

Similarly, the present invention relates to an antibiotic for the treatment of a bacterial pneumonia in a subject,
wherein said antibiotic is selected from the group consisting of macrolide antibiotics such as azithromycin and clarithromycin, fluoroquinolones such as ciprofloxacin and levofloxacin, tetracycline antibiotics such as doxycycline and tetracycline, and
wherein said subject is treated with said antibiotic if it has been determined to have an atypical bacterial pneumonia in the subject with a method according to the invention.

In all the aspects of the present invention, the term “sample” is a biological sample that is obtained from the subject. “Sample” as used herein may, e.g., refer to a sample of bodily fluid or tissue obtained for the purpose of diagnosis, prognosis, or evaluation of a subject of interest, such as a patient. Preferably herein, the sample is a sample of a bodily fluid, such as blood, serum, plasma, urine, saliva, sputum, tears, sweat, nasal secretion and bronchoalveolar lavage (BAL). Particularly, the sample is blood, blood plasma, blood serum, or urine. The samples could be processed (pre-treated), such as by fractionation or purification procedures, for example, separation of whole blood into serum or plasma components. Such pre-treatments can also include, but are not limited to dilution, filtration, centrifugation, concentration, sedimentation, precipitation or dialysis. Pre-treatments may also include the addition of chemical or biochemical substances to the solution, such as acids, bases, buffers, salts, solvents, reactive dyes, detergents, emulsifiers, chelators. Preferably, the sample is a blood sample, more preferably a serum sample or a plasma sample.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood), for example for at least 15 minutes at 2000 to 3000 g.

“Serum” in the context of the present invention is the liquid fraction of whole blood that is collected after the blood is allowed to clot. When coagulated blood (clotted blood) is centrifuged serum can be obtained as supernatant.

As used herein, “urine” is a liquid product of the body secreted by the kidneys through a process called urination (or micturition) and excreted through the urethra.

In those aspects and embodiments of the present invention in which more than one marker is determined in a sample of a subject, the at least two markers are typically but not necessarily determined in the same sample.

As used herein, “histone” or “histone protein”, or “histones” or “histone proteins” refers to the canonical histone(s), such as H1, H2A, H2B, H3 or H4, as well as histone variant(s), such as H3.3, H2A.Z etc. or fragment(s) thereof. Histones form the octamer particle around which DNA is wrapped in order to assemble the chromatin structure (Luger, Nature. 1997 Sep 18; 389(6648):251-60). For example, the histone proteins H2A, H2B, H3, and H4 (two of each) form an octamer, which is wrapped by 165 base pairs of DNA to form the fundamental subunit of chromatin, the nucleosome. Hence, although in the example of the present invention H4 has been determined, the other histone proteins can also be determined. Therefore, in one aspect the at least one histone herein can be selected from the group consisting of H1, H2B, H4, H2A and H3. Accordingly, the level of the histone to be determined in the methods and kits of this aspect of the invention is particularly a level of the histones(s) H1, H2B, H4, H2A and/or H3. In another aspect, the at least one histone herein can be selected from the group consisting of H1, H2A, H2B, H3 and H4. Accordingly, the level of the histone to be determined in the methods and kits of this aspect of the invention is particularly a level of the histones(s) H1, H2A, H2B, H3 and/or H4. The structure of histones and the sequences of the histone proteins are known to the skilled person (Porto & Stein, Front Immunol. (2016) 7: 311).

Exemplary sequences of the histones are given in SEQ ID NOs: 4 to 8. The exemplary amino acid sequence of histone H4 is given in SEQ ID NO: 4. The exemplary amino acid sequence of histone H2A is given in SEQ ID NO: 5. The exemplary amino acid sequence of histone H3 is given in SEQ ID NO: 6. The exemplary amino acid sequence of histone H2B is given in SEQ ID NO: 7. The exemplary amino acid sequence of histone H1 is given in SEQ ID NO: 8. Particularly, the at least one histone is selected from the group consisting of H2B, H4, H2A, H1 and H3. More particularly, the at least one histone is selected from the group consisting of H2B, H4 and H2A. More particularly, the at least one histone is H2B and H4. More particularly, the at least one histone is H2B or H4.

As used herein, the term “proadrenomedullin” or “proADM” refers to proadrenomedullin or a fragment thereof, particularly MR-proADM. It is understood that “determining the level of proADM” or the like refers to determining proADM or a fragment thereof. The fragment can have any length, e.g. at least about 5, 10, 20, 30, 40, 50 or 100 amino acids, so long as the fragment allows the unambiguous determination of the level of the proADM. In particular preferred aspects of the invention, “determining the level of proADM” refers to determining the level of midregional proadrenomedullin (MR-proADM). MR-proADM is a fragment of proADM. The peptide adrenomedullin (ADM) was discovered as a hypotensive peptide comprising 52 amino acids, which had been isolated from a human phenochromocytomeby (Kitamura et al., 1993). Adrenomedullin (ADM) is encoded as a precursor peptide comprising 185 amino acids (“preproadrenomedullin” or “pre-proADM”; SEQ ID NO:9). An exemplary amino acid sequence of pre-proADM is given in SEQ ID NO: 9. ADM comprises the positions 95-146 of the pre-proADM amino acid sequence and is a splice product thereof. “Proadrenomedullin” (“proADM”) refers to pre-proADM without the signal sequence (amino acids 1 to 21), i.e. to amino acid residues 22 to 285 of pre-proADM. “Midregional proadrenomedullin” (“MR-proADM”) refers to the amino acids 42 95 of pre proADM. An exemplary amino acid sequence of MR-proADM is given in SEQ ID NO: 10 It is also envisaged herein that a peptide and fragment thereof of pre proADM or MR-proADM can be used for the herein described methods. For example, the peptide or the fragment thereof can comprise the amino acids 22-41 of pre-proADM (PAMP peptide) or amino acids 95-146 of pre-proADM (mature adrenomedullin). A C-terminal fragment of proADM (amino acids 153 to 185 of preproADM) is called adrenotensin. Fragments of the proADM peptides or fragments of the MR-proADM can comprise, for example, at least about 5, 10, 20, 30 or more amino acids. Accordingly, the fragment of proADM may, for example, be selected from the group consisting of MR-proADM, PAMP, adrenotensin and mature adrenomedullin, preferably herein the fragment is MR-proADM.

High-Mobility-Group-Protein B1 (HMGB1) is like the histone proteins among the most important chromatin proteins. HMGB1 (Uniprot ID P09429; https://www.uniprot.org/uniprot/P09429) is encoded by the HMGB1 gene (NCBI Gene ID: 3146). An exemplary amino acid sequence of a human HMGB1 isoform is given in SEQ ID NO: 11. HMGB1 is known to be upregulated in certain RTIs (Zhou et al., Microbiol. Immunol. (2011) 55:279-288; Patel et al., mBio 9(2):e00246-18).

Fetuin A (FetA) is also known as alpha-2-HS-glycoprotein (AHSG) (Uniprot ID P02765; https://www.uniprot.org/uniprot/P02765) and is in humans encoded by the AHSG gene (NCBI Gene ID: 197). An exemplary amino acid sequence of human Fetuin A is given in SEQ ID NO: 12.

Insulin-like growth factor binding protein, acid labile subunit (IGFALS) (Uniprot ID P35858; https://www.uniprot.org/uniprot/P35858) and is in humans encoded by the IGFALS gene (NCBI Gene ID: 3483). An exemplary amino acid sequence of human IGFALS is given in SEQ ID NO: 13.

C-X-C motif chemokine 10 (CXCL10) (Uniprot ID P02778; https://www.uniprot.org/uniprot/P02778) also known as Interferon gamma-induced protein 10 (IP-10 or IP10) is an 8.7 kDa protein that in humans is encoded by the CXCL10 gene (NCBI Gene ID: 3627). CXCL10 has been shown to be elevated in viral infections (van der Does et al., J Infect. (2016) 72(6):761-763; WO 2016/092554).

Interferon-induced GTP-binding protein Mx1 also known as MX dynamin like GTPase 1 (Uniprot ID P20591; https://www.uniprot.org/uniprot/P20591) is a protein that in humans is encoded by the MX1 gene (NCBI Gene ID: 4599). Mx1 has been proposed as a marker for viral infections (WO 2013/117746, WO 2014/137858).

Serum amyloid A1 (SAA1) (Uniprot ID: P0DJI8; https://www.uniprot.org/uniprot/P0DJI8) is a protein that in humans is encoded by the SAA1 gene (NCBI Gene ID: 6288). SAA1 has been proposed as a marker for tuberculosis (which is not an LRTI in the context of the present invention; M. tuberculosis is not a bacterium causing atypical pneumonia in the context of the present invention) vs. pneumonia, healthy subjects and COPD patients (Jiang et al., PLoS One (2017) 12(3):e0173304).

TNF-related apoptosis-inducing ligand (TRAIL) (Uniprot ID: P50591; https://www.uniprot.org/uniprot/P50591), also known as CD253 and TNFSF10 is a protein that in humans is encoded by the TNFSF10 gene (NCBI Gene ID: 8743). TRAIL has been shown to be elevated in viral infections (van der Does et al., J Infect. (2016) 72(6):761-763; WO 2016/092554, WO 2018/011796, WO 2013/117746, US 10209260, WO 2018/011795, US20190041388).

Procalcitonin (PCT) is a peptide precursor of the hormone calcitonin and has 116 amino acids. PCT has been implicated in the diagnosis of infections or inflammatory diseases of the airways and lungs with associated heart failure, wherein the marker procalcitonin or a partial sequence thereof is determined in a patient to be examined (WO 2008/040328 A2). Methods for its detection (or fragments thereof) are also described in WO 2000/022439 A2 and WO 2008/104321 A1. PCT as a marker in the differential diagnosis of pneumonia has been described (Self et al., Clinical Infectious Diseases (2017) 65(2): 183-90; Neeser et al., Clin Chem Lab Med. (2019) 57(10):1638-1646). PCT assays are commercially available, e.g. the B·R·A·H·M·S PCT sensitive KRYPTOR assay (BRAHMS GmbH, Hennigsdorf, Germany).

As used herein, the “subject” (or “patient”) may be a vertebrate. In the context of the present invention, the term “subject” includes both humans and animals, particularly mammals, and other organisms. Thus, the herein provided methods are applicable to both human and animal subjects. Accordingly, said subject may be an animal such as a mouse, rat, hamster, rabbit, guinea pig, ferret, cat, dog, chicken, sheep, bovine species, horse, camel, or primate. Preferably, the subject is a mammal. Most preferably, the subject is human.

The level of the marker or marker panels, e.g. the at least one histone, IGFALS, HMGB1, MR-proADM, Mx1, PCT, TRAIL, CXCL10 and/or Fetuin A, can be determined by any assay that reliably determines the concentration of the marker(s). Particularly, mass spectrometry (MS) and/or immunoassays can be employed as exemplified in the appended examples. As used herein, an immunoassay is a biochemical test that measures the presence or concentration of a macromolecule/polypeptide in a solution through the use of an antibody or antibody binding fragment or immunoglobulin.

As used herein, the term, “antibody” refers to immunoglobulin molecules and immunologically active portions of immunoglobulin (Ig) molecules, i.e., molecules that contain an antigen binding site that specifically binds (immuno reacts with) an antigen. According to the invention, the antibodies may be monoclonal as well as polyclonal antibodies. Particularly, antibodies that are specifically binding to a marker of interest are used. An antibody is considered to be specific, if its affinity towards the marker of interest, is at least 50-fold higher, preferably 100-fold higher, most preferably at least 1000-fold higher than towards other molecules comprised in a sample containing the molecule of interest. It is well known in the art how to develop and to select antibodies with a given specificity. In the context of the invention, monoclonal antibodies are preferred. Further, antibodies or antigen- binding fragments thereof are used in the methods of the invention that bind specifically to the marker(s) of interest.

Alternatively, instead of antibodies, other capture molecules or molecular scaffolds that specifically and/or selectively recognize target sequences, epitopes, and structural conformations of target proteins may be encompassed by the scope of the present invention. Herein, the term “capture molecules” or “molecular scaffolds” comprises molecules which may be used to bind target molecules or molecules of interest, i.e. analytes (i.e. the markers), from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may, for instance, be mediated by ionic, van-der-Waals, pi-pi, sigma-pi, hydrophobic or hydrogen bond interactions or a combination of two or more of the aforementioned interactions or covalent interactions between the capture molecules or molecular scaffold and the target molecules or molecules of interest. In the context of the present invention, capture molecules or molecular scaffolds may for instance be selected from the group consisting of a nucleic acid molecule, a carbohydrate molecule, a PNA molecule, a protein, a peptide and a glycoprotein. Capture molecules or molecular scaffolds include, for example, aptamers, DARpins (Designed Ankyrin Repeat Proteins), Affimers and the like.

Exemplary immunoassays can be luminescence immunoassay (LIA), radioimmunoassay (RIA), chemiluminescence- and fluorescence- immunoassays, enzyme immunoassay (EIA), Enzyme-linked immunoassays (ELISA), luminescence-based bead arrays, magnetic beads based arrays, protein microarray assays, rapid test formats, rare cryptate assay. Further, assays suitable for point-of-care testing and rapid test formats such as for instance immune-chromatographic strip tests can be employed.

In certain embodiments of an immunoassay employing two antibodies for a marker of interest, one of the antibodies can be labeled and the other antibody can be bound to a solid phase or can be bound selectively to a solid phase. In a particularly preferred aspect of the assay, one of the antibodies is labeled while the other is either bound to a solid phase or can be bound selectively to a solid phase. The first antibody and the second antibody can be present dispersed in a liquid reaction mixture, and wherein a first labelling component which is part of a labelling system based on fluorescence or chemiluminescence extinction or amplification is bound to the first antibody, and a second labelling component of said labelling system is bound to the second antibody so that, after binding of both antibodies to the marker to be detected, a measurable signal which permits detection of the resulting sandwich complexes in the measuring solution is generated. The labelling system can comprise a rare earth cryptate or chelate in combination with a fluorescent or chemiluminescent dye, in particular of the cyanine type.

In a particular embodiment, the method is executed as heterogeneous sandwich immunoassay, wherein one of the antibodies is immobilized on an arbitrarily chosen solid phase, for example, the walls of coated test tubes (e.g. polystyrol test tubes; coated tubes; CT) or microtiter plates, for example composed of polystyrol, or to particles, such as for instance magnetic particles, whereby the other antibody has a group resembling a detectable label or enabling for selective attachment to a label, and which serves the detection of the formed sandwich structures. A temporarily delayed or subsequent immobilization using suitable solid phases is also possible.

The method according to the present invention can furthermore be embodied as a homogeneous method, wherein the sandwich complexes formed by the antibody/antibodies and the marker, which is to be detected remains suspended in the liquid phase. In this case it is preferred, that when two antibodies are used, both antibodies are labeled with parts of a detection system, which leads to generation of a signal or triggering of a signal if both antibodies are integrated into a single sandwich. Such techniques are to be embodied in particular as fluorescence enhancing or fluorescence quenching detection methods. A particularly preferred aspect relates to the use of detection reagents which are to be used pair-wise, such as for example the ones which are described in US 4 882 733 A, EP-B1 0 180 492 or EP-B1 0 539 477 and the prior art cited therein. In this way, measurements in which only reaction products comprising both labeling components in a single immune-complex directly in the reaction mixture are detected, become possible. For example, such technologies are offered under the brand names TRACE® (Time Resolved Amplified Cryptate Emission) or KRYPTOR®, implementing the teachings of the above-cited applications. Therefore, in particular preferred aspects, a diagnostic device is used to carry out the herein provided method.

Further, the immunoassay methods of the present invention may preferably utilize a first antibody and/or a second antibody or antigen-binding fragment(s) or derivative(s) thereof being specific for (an) epitope(s) of the marker to be detected.

Moreover, a host marker or an infectious pathogen (e.g. see US 9,074,236) can be determined by mass spectrometric based methods, such as methods determining the relative quantification or determining the absolute quantification of the marker of interest. The MS technology or other detection method such as molecular based methods can be combined with immunological tests. Relative quantification “rSRM” may e.g. be achieved by:
1. Determining increased or decreased presence of the target protein by comparing the SRM (Selected reaction monitoring) signature peak area from a given target fragment peptide detected in the sample to the same SRM signature peak area of the target fragment peptide in at least a second, third, fourth or more biological samples.
2. Determining increased or decreased presence of target protein by comparing the SRM signature peak area from a given target peptide detected in the sample to SRM signature peak areas developed from fragment peptides from other proteins, in other samples derived from different and separate biological sources, where the SRM signature peak area comparison between the two samples for a peptide fragment are normalized for e.g. to amount of protein analyzed in each sample.
3. Determining increased or decreased presence of the target protein by comparing the SRM signature peak area for a given target peptide to the SRM signature peak areas from other fragment peptides derived from different proteins within the same biological sample in order to normalize changing levels of the biomarker to levels of other proteins that do not change their levels of expression under various cellular conditions.
4. These assays can be applied to both unmodified fragment peptides and to modified fragment peptides of the target proteins, where the modifications include, but are not limited to phosphorylation and/or glycosylation, acetylation, methylation (mono, di, tri), citrullination, ubiquitinylation and where the relative levels of modified peptides are determined in the same manner as determining relative amounts of unmodified peptides.

Absolute quantification of a given peptide may be achieved by:
1. Comparing the SRM/MRM signature peak area for a given fragment peptide from the target proteins in an individual biological sample to the SRM/MRM signature peak area of an internal fragment peptide standard spiked into the protein lysate from the biological sample. The internal standard may be a labeled synthetic version of the fragment peptide from the target protein that is being interrogated or the labeled recombinant protein. This standard is spiked into a sample in known amounts before (mandatory for the recombinant protein) or after digestion, and the SRM/MRM signature peak area can be determined for both the internal fragment peptide standard and the native fragment peptide in the biological sample separately, followed by comparison of both peak areas. This can be applied to unmodified fragment peptides and modified fragment peptides, where the modifications include but are not limited to phosphorylation and/or glycosylation, acetylation, methylation (e.g. mono-, di-, or tri-methylation), citrullination, ubiquitinylation, and where the absolute levels of modified peptides can be determined in the same manner as determining absolute levels of unmodified peptides.
2. Peptides can also be quantified using external calibration curves. The normal curve approach uses a constant amount of a heavy peptide as an internal standard and a varying amount of light synthetic peptide spiked into the sample. A representative matrix similar to that of the test samples needs to be used to construct standard curves to account for a matrix effect. Besides, reverse curve method circumvents the issue of endogenous analyte in the matrix, where a constant amount of light peptide is spiked on top of the endogenous analyte to create an internal standard and varying amounts of heavy peptide are spiked to create a set of concentration standards. Test samples to be compared with either the normal or reverse curves are spiked with the same amount of standard peptide as the internal standard spiked into the matrix used to create the calibration curve.

Further diagnostic methods can be additionally used for the improvement of the management of the patient, clinical decision making or the monitoring of the infection by identifying pathologic strains, important mutations as well antibiotic. This before mentioned methods can be molecular based technologies such as (Real-Time) Polymerase Chain Reaction (RT-PCT) or the Next Generation Sequencing (NGS), Mass Spectrometry (MS) as well as culturing-based applications. These further interventions can be tested simultaneously or at another time point.

All here before mentioned diagnostic methods can be done from one or from further samples of the patient like in serial measurement.

The term Real-Time PCR is intended to mean any amplification technique which makes it possible to monitor the progress of an ongoing amplification reaction as it occurs (i.e. in real time). Data is therefore collected during the exponential phase of the PCR reaction, rather than at the end point as in conventional PCR. Measuring the kinetics of the reaction in the early phases of PCR provides distinct advantages over traditional PCR detection. In real-time PCR, reactions are characterized by the point in time during cycling when amplification of a target is first detected rather than the amount of target accumulated after a fixed number of cycles. The higher the starting copy number of the nucleic acid target, the sooner a significant increase in fluorescence is observed. Traditional PCR methods may also be applied, and use separation methods, such as agarose gels, for detection of PCR amplification at the final phase of or endpoint of the PCR reaction. For qRT-PCR no post-PCR processing of the unknown DNA sample is necessary as the quantification occurs in real-time during the reaction. Furthermore, an increase in reporter fluorescent signal is directly proportional to the number of amplicons generated. As the method was designed to use similar experimental conditions, the PCR amplification for each multiplex can be performed using the same thermal cycling profile thereby allowing the amplification of all the nucleic acid targets at the same time in a single apparatus (e.g. thermocycler).

Although nucleic acid amplification is often performed by PCR or RT-PCR, other methods exist. Non-limiting examples of such method include quantitative polymerase chain reaction (Q-PCR), digital droplet PCR (ddPCR), ligase chain reaction (LCR), transcription-mediated amplification (TMA), self-sustained sequence replication (3SR), nucleic acid sequence-based amplification (NASBA), strand displacement amplification (SDA), recombinase polymerase amplification (RPA), loop-mediated isothermal amplification (LAMP), helicase-dependent amplification (HDA), helicase-dependent isothermal DNA amplification (tHDA), branched DNA (bDNA), cycling probe technology (CPT), solid phase amplification (SPA), rolling circle amplification technology (RCA), real-time RCA, solid phase RCA, RCA coupled with molecular padlock probe (MPP/RCA), aptamer based RCA (aptamer-RCA), anchored SDA, primer extension preamplification (PEP), degenerate oligonucleotide primed PCR (DOP-PCR), sequence-independent single primer amplification (SISPA), linker-adaptor PCR, nuclease dependent signal amplification (NDSA), ramification amplification (RAM), multiple displacement amplification 5 (MDA), real-time RAM, and whole genome amplification (WGA) (Westin, L. et al., 2000, Nat. Biotechnol. 18: 199-204; Notomi, T. et al., 2000, Nucleic Acids Res. 28:e63; Vincent, M. et al., 2004, EMBO reports 5:795-800; Piepenburg, O. et al., 2006, PLoS Biology 4:E204; Yi, J. et al., 2006, Nucleic Acids Res. 34:e81; Zhang, D. et al., 2006, Clin. Chim. Acta 363:61-70; McCarthy, E. L. et al., 2007, Biosens.Biotechnol. 22:126-1244; Zhou, L. et al., 2007, Anal. Chem. 79:7492-7500; Coskun, S. and Alsmadi, O., 2007, Prenat. Diagn. 27:297-302; Biagini, P. et al., 2007, J. Gen. Virol. 88:2629- 2701; Gill, P. et al., 2007, Diagn. Microbiol. Infect. Dis. 59:243-249; Lasken, R. S. and Egholm, M., 2003, Trends Biotech. 21:531-535).

It should also be understood herein that the scope of the invention is not limited to a specific detection technology and that in the context of the present invention different technologies such as immunoassays, PCR and MS can be combined.

The sensitivity and specificity of a diagnostic or prognostic methods as the methods of the present invention depends on more than just the analytical quality of the test, it also depends on the definition of what constitutes a specific result, e.g. an abnormal (diseased) or normal (healthy) result. The distribution of levels of the marker(s), for subjects with and without a certain condition (e.g. RTI/LRTI, typical/atypical pneumonia, bacterial/viral, healthy/diseased), might overlap. Under such conditions, a test does not absolutely distinguish subjects with and without a specific condition with 100% accuracy. In other words, a balance between the inclusion of false negative and false positive results has to be found. The skilled person is aware of the fact that the physical condition per se of a subject or at least one further maker and/or parameter of the subject can assist in the interpretation of the data and that this further information allows a more reliable diagnosis in the areas of overlap.

In practice, Receiver Operating Characteristic curves (ROC curves), are typically calculated by plotting the value of a variable versus its relative frequency in “normal” (e.g. apparently healthy individuals not having a prenatal disorder or condition) and “disease” populations (similarly e.g. for two different states like viral and bacterial RTI or typical and atypical pneumonia). For any particular marker, a distribution of marker levels for subjects with and without a disease/condition will likely overlap. Under such conditions, a test does not absolutely distinguish normal from disease with 100% accuracy, and the area of overlap might indicate where the test cannot distinguish normal from disease. A threshold is selected, below which the test is considered to be “abnormal” and above which the test is considered to be “normal” or below or above which the test indicates a specific condition. The area under the ROC curve (AUC) is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art; see, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/- 5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve (AUC) can be used to determine the effectiveness of the test.

In other embodiments, a positive likelihood ratio, negative likelihood ratio, odds ratio, or hazard ratio is used as a measure of a test’s ability to predict risk or diagnose a disorder or condition (“diseased group”). In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group.

In the case of a hazard ratio, a value of 1 indicates that the relative risk of an endpoint (e.g., death or a specific outcome) is equal in both the “diseased” and “control” groups; a value greater than 1 indicates that the risk is greater in the diseased group; and a value less than 1 indicates that the risk is greater in the control group. “Diseased” and “control” groups herein are representative for two groups of different condition.

The skilled artisan will understand that associating a diagnostic or prognostic indicator, with a diagnosis or with a prognostic risk of a future clinical outcome is a statistical analysis. For example, a marker level of lower than X may signal that a patient is more likely to suffer from an adverse outcome than patients with a level more than or equal to X, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value; see, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

As outlined herein above, in addition to the particular marker(s) or marker panels to be detected in the aspects of the invention, further parameters can be taken into account for a particular diagnosis or differential diagnosis. As used herein, a parameter is a characteristic, feature, or measurable factor that can help in defining a particular system. A parameter is an important element for health- and physiology-related assessments, such as a disease/disorder/clinical condition risk. Furthermore, a parameter is defined as a characteristic that is objectively measured and evaluated as an indicator of normal biological processes, pathogenic processes, or pharmacologic responses to a therapeutic intervention.

An exemplary parameter can be selected from the group consisting of body mass index, weight, age, sex, diagnostic scores, results from imaging methods such as x-ray, white blood cell count, body temperature, blood pressure, respiratory rate, heart rate, oxygen saturation, breathing sounds and smoking behavior.

The invention furthermore relates to a “kit” or the use of such a kit for in vitro diagnosis of RTI/LRTI/pneumonia, where a determination of at least one marker selected from the group of HMGB1, histone protein, IGFALS and Fetuin A is carried out in a subject to be investigated, particularly in a method according to the invention. The kit comprises detection reagents comprising capture molecules like antibodies, and optionally further reagents such as buffers and/ or calibrators. The following markers and combinations of markers are preferred (i.e. the kits comprise detection reagents for the following combinations or markers):
HMGB1;
HMGB1 + MR-proADM;
HMGB1 + FetA;
HMGB1 + TRAIL, IP10, PCT and/or MX1;
HMGB1 + PCT, MR-proADM, histone protein (particularly H4), SAA1, FetA, IGFALS, TRAIL and/or CXCL10;
histone protein (particularly H4) + IGFALS;
IGFALS + HMGB1;
IGFALS + TRAIL, IP10, PCT and/or MX1;
IGFALS + MR-proADM;
IGFALS + PCT, MR-proADM, histone protein (particularly H4), SAA1, FetA, TRAIL and/or CXCL10;
histone protein (particularly H4) + TRAIL, IP10, PCT and/or MX1;
histone protein (particularly H4) + PCT, MR-proADM, SAA1, FetA, IGFALS, TRAIL and/or CXCL10.

### Aspects of the Invention

The present invention in particular aspects relates to the following:
1. A method for diagnosing a respiratory tract infection in a subject suspected of having a respiratory tract infection (RTI), comprising
   determining in a sample from said subject the level of High-Mobility-Group-Protein B1 (HMGB 1),
   wherein the subject is diagnosed with a respiratory tract infection when the level of HMGB1 is above a predetermined threshold level.
2. A method for the differential diagnosis of a disease of the respiratory tract in a subject, comprising
   determining in a sample from said subject the level of High-Mobility-Group-Protein B1 (HMGB 1),
   wherein the subject is diagnosed with a respiratory tract infection (RTI) when the level of HMGB1 is above a predetermined threshold level.
3. The method according to aspect 1 or 2, wherein the subject has one or more symptoms of a lower respiratory tract infection (LRTI).
4. The method of aspect 3, wherein said subjects shows one or more symptoms selected from shortness of breath, weakness, fever, sputum formation, coughing, fatigue, wheezing, chest discomfort or pain, rapid breathing, difficulty breathing, congestion, running nose and sore throat.
5. The method of aspect 4, wherein said subject suffers from cough and one or more symptoms selected from sputum formation, shortness of breath, wheezing and chest discomfort or pain.
6. The method of any of the preceding aspects, wherein the RTI is a lower respiratory tract infection (LRTI).
7. The method of aspect 6 wherein the LRTI is selected from the group of acute bronchitis, pneumonia and bronchiolitis.
8. The method of aspect 7, wherein the LRTI is a bacterial or a viral infection.
9. The method of aspect 8, wherein the LRTI is an atypical bacterial infection, particularly an atypical bacterial pneumonia.
10. The method of aspect 8, wherein the viral infection is selected from the group consisting of influenza virus such as Influenza A or Influenza B, respiratory syncytial virus (RSV), coronavirus, particularly Severe acute respiratory syndrome (SARS), Middle East respiratory syndrome (MERS) or Coronavirus disease 2019 (COVID-19), rhinovirus, parainfluenza viruses, human metapneumovirus, varicella, hantavirus and adenovirus .
11. The method of any of the preceding aspects, wherein additionally the level of one or more further markers selected from the group consisting of procalcitonin (PCT), proadrenomedullin (proADM) or a fragment thereof, histone protein, Serum amyloid A1 (SAA1), Fetuin-A (FetA), Insulin-like growth factor binding protein, acid labile subunit (IGFALS), Tumor Necrosis Factor Related Apoptosis Inducing Ligand (TRAIL) and C-X-C motif chemokine 10 (CXCL10) is determined in a sample from said subject.
12. The method of aspect 11, wherein the level of MR-proADM is determined in a sample from said subject, preferably wherein the level of MR-proADM is indicative for the severity of the infection.
13. The method of any of the preceding aspects, wherein additionally the level of one or histone proteins selected from histone H2B, histone H4, histone H2A, histone H3 and histone H1 is determined in a sample from said subject, preferably the level of H4 is determined, and
   wherein the subject is diagnosed with atypical bacterial pneumonia when the level of the at least one histone protein, preferably H4, is above a predetermined threshold level, and/or
   wherein the subject is diagnosed with typical bacterial pneumonia when the level of the at least one histone protein, preferably H4, is above a first predetermined threshold level and below a second predetermined threshold level.
14. The method of any of the preceding aspects, wherein the sample is a sample of a bodily fluid, preferably a blood sample, a saliva sample, nasal swab, sweat, a urine sample or a bronchoalveolar lavage (BAL), more preferably serum, plasma or whole blood, most preferably plasma.
15. An antibiotic for use in the treatment of a bacterial respiratory tract infection in a subject, wherein said subject is treated with the antibiotic if it has been determined to have a bacterial respiratory tract infection with the method according to aspect 13.
16. A method for diagnosing a respiratory tract infection in a subject, comprising determining in a sample from said subject the level of a histone protein, preferably selected from histone H4, histone H2A, histone H2B, histone H3 and histone H1, and/or determining in a sample from said subject the level of Insulin-like growth factor binding protein, acid labile subunit (IGFALS),
   wherein the subject is diagnosed with a bacterial respiratory tract infection when the level of IGFALS is below a predetermined threshold level and/or the level of the histone protein is above a predetermined threshold value.
17. The method of aspect 16, wherein the histone protein is H4.
18. The method of aspects 16 and 17, wherein said subject is suspected of having a bacterial respiratory tract infection.
19. The method of aspect 18, wherein said subject has one or more symptoms of a lower respiratory tract infection (LRTI), particularly pneumonia.
20. The method of aspect 19, wherein said subjects shows one or more symptoms selected from shortness of breath, weakness, fever, sputum formation, coughing, fatigue, wheezing, chest discomfort or pain, rapid breathing, difficulty breathing, congestion, running nose and sore throat.
21. The method of aspect 20, wherein said subject suffers from cough and one or more symptoms selected from sputum formation, shortness of breath, wheezing and chest discomfort or pain.
22. The method of aspects 16 to 21, wherein additionally the level of one or more further markers selected from the group consisting of procalcitonin (PCT), proadrenomedullin (proADM) or a fragment thereof, histone protein, High-Mobility-Group-Protein B1 (HMGB 1), Serum amyloid A1 (SAA1), Fetuin-A (FetA), Tumor Necrosis Factor Related Apoptosis Inducing Ligand (TRAIL) and C-X-C motif chemokine 10 (CXCL10) is determined in a sample from said subject.
23. The method of aspect 22, wherein the level of MR-proADM is determined in a sample from said subject, preferably wherein the level of MR-proADM is indicative for the severity of the infection.
24. The method of aspects 26 to 23, wherein the level of High-Mobility-Group-Protein B1 (HMGB1) is determined in a blood sample from said subject, and wherein the subject is diagnosed with a bacterial respiratory tract infection when the level of IGFALS is below a predetermined threshold level, the level of the histone protein is above a predetermined threshold value and the level of HMGB1 is above a predetermined threshold level.
25. The method of aspects 16 to 24, wherein the subject is diagnosed with atypical bacterial pneumonia when the level of the at least one histone protein, preferably H4, is above a predetermined threshold level.
26. The method of aspects 16 to 25, wherein the subject is diagnosed with typical bacterial pneumonia when the level of the at least one histone protein, preferably H4, is above a first predetermined threshold level and below a second predetermined threshold level.
27. The method of aspects 16 to 26, wherein the sample is a sample of a bodily fluid, preferably a blood sample, a saliva sample, nasal swab, sweat sample, a urine sample or a bronchoalveolar lavage (BAL), more preferably serum, plasma or whole blood, most preferably plasma.
28. An antibiotic for use in the treatment of a bacterial respiratory tract infection in a subject, wherein said subject is treated with the antibiotic if it has been determined to have a bacterial respiratory tract infection with the method according to aspects 16 to 27.

### SEQUENCES

Exemplary sequence listings for the markers of the present invention are given in the appended sequence protocol. The following sequences are included in the sequence protocol:
SEQ ID NO 1: Peptide fragment of histone H4 detected by mass spectrometry.
SEQ ID NO 2: Peptide fragment of Fetuin A detected by mass spectrometry.
SEQ ID NO 3: Peptide fragment of SAA1 detected by mass spectrometry.
SEQ ID NO 4: Amino acid sequence of histone H4.
SEQ ID NO 5: Amino acid sequence of histone H2A.
SEQ ID NO 6: Amino acid sequence of histone H3.
SEQ ID NO 7: Amino acid sequence of histone H2B.
SEQ ID NO 8: Amino acid sequence of histone H1.
SEQ ID NO 9: Amino acid sequence of pre-proADM.
SEQ ID NO 10: Amino acid sequence of MR-proADM.
SEQ ID NO 11: Amino acid sequence of human HMGB1.
SEQ ID NO 12: Amino acid sequence of human Fetuin A.
SEQ ID NO 13: Amino acid sequence of human IGFALS.

### EXAMPLES

### Methods

Biomarker proteins were quantified in samples from different patient populations from different hospitals. Different biomarker levels were analyzed in patients who suffered from respiratory tract infections (RTI) including viral RTI, bacterial RTI, typical bacterial pneumonia and atypical bacterial pneumonia. In addition, the biomarker levels were measured in samples from non-infected patients with RTI-like symptoms (RTI “mimics”), pneumonia-like symptoms (pneumonia mimics) and healthy patients.

Patient enrollment and sample draw were conducted in the emergency department and samples were categorized as viral or bacterial infection following the subsequent methods:

In patients with suspected viral infection the viral infection has been confirmed by molecular testing. In patients with suspected bacterial infections the bacterial infection has been proven by a positive bacterial culture result or by a positive bacterial antigen-testing. Specific pathogen biomolecules were detected either in blood, lung specimen or urine (Legionella antigen assay). Two patients with mixed bacterial and viral infections have been excluded from the evaluation. Only samples from patients with confirmed presence of a viral or bacterial infection were included for further testing.

For the purpose of comparison between different types of pneumonia, samples from patients with proven typical and atypical pathogens have been collected. Typical pneumonia cases have been diagnosed following the local pathway for pneumonia diagnosis. Only those patients with pneumonia specific symptoms and a positive typical pathogen identification in a respiratory tract derived sample were included as typical pneumonia patients. Atypical pneumonia has been diagnosed in patients with pneumonia symptoms by molecular methods or by detection of specific atypical antigens in urine.

The so-called RTI mimics group is characterized by samples from non-(bacterial) infected patients with overlapping RTI symptoms as for example dyspnea, cough or chest pain. The samples were collected from patients e.g. diagnosed with Heart Failure, Asthma and COPD. The other so-called Pneumonia mimics group is also characterized by samples from non-infected patients with ACS, Asthma, COPD, Heart Failure, Lung Embolism, Tumor of the lung or other Tumors or Atrial Fibrillation. Patients with proven bacterial infection were excluded from the RTI or pneumonia mimics group.

TABLE 1

Patient characteristics

| Patient characteristics | Number of patients |
|---|---|
| RTI viral | 30 |
| RTI bacterial | 31 |
| Bacterial pneumonia, atypical | 27 |
| Bacterial pneumonia, typical | 31 |
| RTI mimics with viral Bronchitis | 25 |
| RTI mimics with Chronic Obstructive Pulmonary Disease (COPD) | 25 |
| RTI mimics with Heart Failure (HF) | 15 |
| RTI mimics with Asthma | 13 |
| Pneumonia mimics with Acute Coronary Syndrome (ACS) | 8 |
| Pneumonia mimics with Asthma | 15 |
| Pneumonia mimics with Chronic Obstructive Pulmonary Disease (COPD) | 22 |
| Pneumonia mimics with Heart Failure (HF) | 15 |
| Pneumonia mimics with Lung Embolism | 6 |
| Pneumonia mimics with Tumors of the Lung or other Tumors | 9 |
| Pneumonia mimics with atrial fibrillation | 3 |
| Healthy | 40 |

### Biomarkers

Determination by immuno assays:
IGFALS (Insulin-like growth factor-binding protein complex acid labile subunit) levels were measured by an Enzyme-linked Immunosorbent Assay (ALS human ELISA) from BioVendor, Czech Republic.
HMGB1 (High-mobility group box 1) values were determined by an Enzyme-linked Immunosorbent Assay (HMGB 1 ELISA) from IBL International, Germany.
MR-proADM (midregional proadrenomedullin) and PCT (procalcitonin) levels were determined in plasma samples using the ultrasensitive assays B.R.A.H.M.S MR-proADM and B.R.A.H.M.S PCT via the KRYPTOR random access analyzer from Thermo Fisher Scientific, Germany.
Fetuin A was measured with the magnetic bead-based multiplex assay of the Luminex platform from R&D Systems, US.
TRAIL (TNF-related apoptosis-inducing ligand) and CXCL10 (interferon-gamma induced protein 10 kD) were also measured with the magnetic bead-based multiplex assay of the Luminex platform from R&D Systems.

### Determination by Mass Spectrometer (MS)

The levels of histone H4 (detected peptide sequence “VFLENVIR”, SEQ ID NO: 1), Fetuin A (detected peptide sequence “FSVVYAK”, SEQ ID NO: 2) and SAA1 (detected peptide sequence “EANYIGSDK”, SEQ ID NO: 3) were determined in the plasma samples by selected reaction monitoring or multiple reaction monitoring (SRM/MRM) assays.

The SRM assays were developed on a triple quadrupole mass spectrometer TSQ Quantiva coupled with HPLC Ultimate 3000 (Thermo Fisher Scientific). The peptides were identified by co-eluting light and heavy-labeled transitions in the chromatographic separation. Pinpoint (Thermo Fisher Scientific) and Skyline (MacCoss Lab) software were used for time alignment, relative quantification of the transitions and targeted protein quantification.

### Statistical Analysis

Biomarker levels < LoD (Limit of Detection) of immunoassays were imputed with half of LoD (appr. 10% of measurements were < LoD). Missing biomarker levels led to the exclusion of patient samples from the respective comparison. Diagnostic accuracy in separating two diagnostic subgroups (e.g. healthy vs. affected) was assessed for all biomarkers that had available data. There was no treatment of statistical outliers in any of the analyses, so all observations received the same weight in the calculations. The area under the receiver operating characteristics curve (AUC), which is independent of cutoffs, served as the primary measure of diagnostic accuracy. All analyses were conducted in R 3.5.1 (R Core Team 2018).

For single biomarkers, AUC was calculated based on the Wilcoxon statistic W belonging to the Wilcoxon rank sum test comparing biomarker levels from two diagnosis subgroups with sample sizes n₁ and n₂, following the formula AUC= W/(n1^{∗}n2).

For dual and three-way biomarker combinations, AUCs were calculated based on the C-statistic of logistic regression models fitted via maximum likelihood estimation. Models contained biomarkers as predictors and the diagnosis subgroup as binary dependent variable. Prior to multivariate modelling, all biomarker levels had been log-transformed after imputing all zeroes with 0.005. Regression models were fit using the R package rms (Frank E Harrell Jr (2019). rms: Regression Modeling Strategies. R package version 5.1-4

ROC plots were created using the R package ROCR (Sing et al., Bioinformatics (2005) 21(20):3940-3941). Boxplots were created using the R package ggplot2 (Wickham, ggplot2: Elegant Graphics for Data Analysis, Springer Verlag (2016)).

TABLE 2

A UC values for different marker combinations for different differential diagnoses

| Marker(s) | AUC | | | | | | | |
|---|---|---|---|---|---|---|---|---|
| | Infection vs. healthy | Pneumonia vs. | healthy | Bacterial pneumonia vs. pneumonia mimic | Typical pneumonia vs. atypical pneumonia | Atypical pneumonia vs. healthy | Atypical pneumonia vs. pneumonia mimic | Atypical pneumonia vs. RTI mimic |
| H4 | 0.793 | 0.954 | | | 0.768 | 0.985 | | |
| H4+PCT | | 0.967 | | | | | | |
| Fetuin A | | 0.778 | | 0.748 | | | 0.711 | |
| IGFALS | | 0.751 | | 0.687 | 0.846 | | 0.529 | |
| HMGB1 | | 0.941 | | | | 0.974 | | 0.996 |
| HMGB1+PCT | | 0.969 | | | | | | |

TABLE 3

C-Index values for the ROC curves of different markers and marker combinations in the differentiation of bacterial vs viral pneumonia

| Marker(s) | C-Index |
|---|---|
| | bacterial vs viral |
| Fetuin A | 0.84 |
| Fetuin A + PCT | 0.86 |
| Fetuin A + Trail | 0.94 |
| IGFALS | 0.79 |
| IGFALS+PCT | 0.80 |
| IGFALS | 0.94 |
| H4+TRAIL | 0.88 |

1. A method for diagnosing a respiratory tract infection in a subject, comprising
determining in a sample from said subject,
(i) the level of High-Mobility-Group-Protein B1 (HMGB1), and/or
(ii) the level of a histone protein, and/or
(iii) the level of Insulin-like growth factor binding protein, acid labile subunit (IGFALS), wherein the subject is diagnosed with a respiratory tract infection (RTI) when the level of HMGB1 is above a predetermined threshold level, and/or
wherein the subject is diagnosed with a bacterial RTI when the level of IGFALS is below a predetermined threshold level, and/or the level of the histone protein is above a predetermined threshold value.

2. The method of claim 1, wherein the subject is suspected of having a RTI.

3. The method of claim 1, wherein the method comprises determining in sample from said subject the level of High-Mobility-Group-Protein B1 (HMGB1), wherein the subject is diagnosed with the respiratory tract infection when the level of HMGB1 is above a predetermined threshold level.

4. A method for the differential diagnosis of a disease of the respiratory tract in a subject, comprising
determining in a sample from said subject the level of High-Mobility-Group-Protein B1 (HMGB1),
wherein the subject is diagnosed with a respiratory tract infection (RTI) when the level of HMGB1 is above a predetermined threshold level.

5. The method of claim 1, wherein the subject has one or more symptoms of a lower respiratory tract infection (LRTI).

6. The method of claim 5, wherein said subject shows one or more symptoms selected from the group consisting of shortness of breath, weakness, fever, sputum formation, coughing, fatigue, wheezing, chest discomfort or pain, rapid breathing, difficulty breathing, congestion, running nose and sore throat.

7. The method of claim 6, wherein said subject suffers from cough and one or more symptoms selected from the group consisting of sputum formation, shortness of breath, wheezing, chest discomfort, and chest pain.

8. The method of claim 1 wherein the RTI is a lower respiratory tract infection (LRTI).

9. The method of claim 8, wherein the LRTI is selected from the group of acute bronchitis, pneumonia and bronchiolitis.

10. The method of claim 9, wherein the LRTI is a bacterial or a viral infection.

11. The method of claim 10, wherein the LRTI is an atypical bacterial infection, particularly an atypical bacterial pneumonia.

12. The method of claim 10, wherein the viral infection is selected from the group consisting of an influenza virus, respiratory syncytial virus (RSV), coronavirus, Middle East respiratory syndrome (MERS), Coronavirus disease 2019 (COVID-19), rhinovirus, parainfluenza virus, human metapneumovirus, varicella, hantavirus and adenovirus infection.

13. The method of claim 1, wherein additionally the level of one or more further markers selected from the group consisting of procalcitonin (PCT), proadrenomedullin (proADM) , a fragment of pro ADM, histone protein, Serum amyloid A1 (SAA1), Fetuin-A (FetA), Insulin-like growth factor binding protein, acid labile subunit (IGFALS), Tumor Necrosis Factor Related Apoptosis Inducing Ligand (TRAIL) and C-X-C motif chemokine 10 (CXCL10) is determined in a sample from said subject.

14. The method of claim 13, wherein the level of MR-proADM is determined in a sample from said subject.

15. The method of claim 3, wherein additionally the level of one or histone proteins selected from the group consisting of histone H2B, histone H4, histone H2A, histone H3 and histone H1 is determined in a sample from said subject, and
wherein the subject is diagnosed with atypical bacterial pneumonia when the level of the at least one histone protein, is above a predetermined threshold level, and/or
wherein the subject is diagnosed with typical bacterial pneumonia when the level of the at least one histone protein, is above a first predetermined threshold level and below a second predetermined threshold level.

16. The method of claim 1, wherein the method comprises determining in a sample from said subject
(i) the level of a histone protein, and/or
(ii) the level of Insulin-like growth factor binding protein, acid labile subunit (IGFALS), wherein the subject is diagnosed with a bacterial respiratory tract infection when the level of IGFALS is below a predetermined threshold level and/or the level of the histone protein is above a predetermined threshold value.

17. The method of claim 16, wherein the method comprises determining the level of a histone protein and wherein the histone protein is selected from histone H4, histone H2A, histone H2B, histone H3 and histone H1.

18. The method of claim 17, wherein the histone protein is H4.

19. The method of claim 16, wherein said subject is suspected of having a bacterial respiratory tract infection.

20. The method of claim 19, wherein said subject has one or more symptoms of a lower respiratory tract infection (LRTI).

21. The method of claim 20, wherein said subjects shows one or more symptoms selected from shortness of breath, weakness, fever, sputum formation, coughing, fatigue, wheezing, chest discomfort, pain, rapid breathing, difficulty breathing, congestion, running nose and sore throat.

22. The method of claim 21, wherein said subject suffers from cough and one or more symptoms selected from the group consisting of sputum formation, shortness of breath, wheezing and chest discomfort and chest pain.

23. The method of claim 16, wherein additionally the level of one or more further markers selected from the group consisting of procalcitonin (PCT), proadrenomedullin (proADM), a fragment of proADM, histone protein, High-Mobility-Group-Protein B1 (HMGB1), Serum amyloid A1 (SAA1), Fetuin-A (FetA), Tumor Necrosis Factor Related Apoptosis Inducing Ligand (TRAIL) and C-X-C motif chemokine 10 (CXCL10) is determined in a sample from said subject.

24. The method of claim 23, wherein the level of MR-proADM is determined in a sample from said subject.

25. The method of claim 16, wherein the level of High-Mobility-Group-Protein B1 (HMGB1) is determined in a blood sample from said subject, and wherein the subject is diagnosed with a bacterial respiratory tract infection when the level of IGFALS is below a predetermined threshold level, the level of the histone protein is above a predetermined threshold value and the level of HMGB1 is above a predetermined threshold level.

26. The method of claim 16, wherein the subject is diagnosed with atypical bacterial pneumonia when the level of the at least one histone protein, preferably H4, is above a predetermined threshold level.

27. The method of claim 16, wherein the subject is diagnosed with typical bacterial pneumonia when the level of the at least one histone protein, preferably H4, is above a first predetermined threshold level and below a second predetermined threshold level.

28. The method of claim 1, wherein the sample is a sample of a bodily fluid.

29. (canceled)


SEQUENCE LISTING (13 sequences)

SEQ ID NO: 1 - protein, 8 aa, Homo sapiens
  peptide fragments of histone H4
VFLENVIR

SEQ ID NO: 2 - protein, 7 aa, Homo sapiens
  peptide fragments of Fetuin A
FSVVYAK

SEQ ID NO: 3 - protein, 9 aa, Homo sapiens
  peptide fragments of SAA1
EANYIGSDK

SEQ ID NO: 4 - protein, 102 aa, Homo sapiens
  amino acid sequence of human histone H4, uniprot ID P62805.2
SGRGKGGKGLGKGGAKRHRKVLRDNIQGITKPAIRRLARRGGVKRISGLIYEETRGVLKVFLENVIRDAVTYTEHAKRKTVTAMDVVYALKRQGRTLYGFGG

SEQ ID NO: 5 - protein, 130 aa, Homo sapiens
  amino acid sequence of human histone H2A type 1, uniprot ID Q96QV6
SGRGKQGGKARAKSKSRSSRAGLQFPVGRIHRLLRKGNYAERIGAGAPVYLAAVLEYLTAEILELAGNASRDNKKTRIIPRHLQLAIRNDEELNKLLGGVTIAQGGVLPNIQAVLLPKKTESHHHKAQSK

SEQ ID NO: 6 - protein, 135 aa, Homo sapiens
  amino acid sequence of human histone H3.1, uniprot ID P68431.2
ARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 7 - protein, 126 aa, Homo sapiens
  amino acid sequence of human histone H2B, uniprot ID P62807
MPEPAKSAPAPKKGSKKAVTKAQKKDGKKRKRSRKESYSVYVYKVLKQVHPDTGISSKAMGIMNSFVNDIFERIAGEASRLAHYNKRSTITSREIQTAVRLLLPGELAKHAVSEGTKAVTKYTSSK

SEQ ID NO: 8 - protein, 215 aa, Homo sapiens
  amino acid sequence of human histone H1, uniprot ID Q02539
MSETVPPAPAASAAPEKPLAGKKAKKPAKAAAASKKKPAGPSVSELIVQAASSSKERGGVSLAALKKALAAAGYDVEKNNSRIKLGIKSLVSKGTLVQTKGTGASGSFKLNKKASSVETKPGASKVATKTKATGASKKLKKATGASKKSVKTPKKAKKPAATRKSSKNPKKPKTVKPKKVAKSPAKAKAVKPKAAKARVTKPKTAKPKKAAPKKK

SEQ ID NO: 9 - protein, 185 aa, Homo sapiens
  amino acid sequence of pre-pro-ADM
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 10 - protein, 48 aa, Homo sapiens
  amino acid sequence of MR-pro-ADM (amino acid residues 45-92 of pre-pro-ADM)
ELRMSSSYPTGLADVKAGPAQTLIRPQDMKGASRSPEDSSPDAARIRV

SEQ ID NO: 11 - protein, 215 aa, Homo sapiens
  Amino acid sequence of human HMGB1
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 12 - protein, 367 aa, Homo sapiens
  Amino acid sequence of human Fetuin A
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 13 - protein, 605 aa, Homo sapiens
  Amino acid sequence of human IGFALS
MALRKGGLALALLLLSWVALGPRSLEGADPGTPGEAEGPACPAACVCSYDDDADELSVFCSSRNLTRLPDGVPGGTQALWLDGNNLSSVPPAAFQNLSSLGFLNLQGGQLGSLEPQALLGLENLCHLHLERNQLRSLALGTFAHTPALASLGLSNNRLSRLEDGLFEGLGSLWDLNLGWNSLAVLPDAAFRGLGSLRELVLAGNRLAYLQPALFSGLAELRELDLSRNALRAIKANVFVQLPRLQKLYLDRNLIAAVAPGAFLGLKALRWLDLSHNRVAGLLEDTFPGLLGLRVLRLSHNAIASLRPRTFKDLHFLEELQLGHNRIRQLAERSFEGLGQLEVLTLDHNQLQEVKAGAFLGLTNVAVMNLSGNCLRNLPEQVFRGLGKLHSLHLEGSCLGRIRPHTFTGLSGLRRLFLKDNGLVGIEEQSLWGLAELLELDLTSNQLTHLPHRLFQGLGKLEYLLLSRNRLAELPADALGPLQRAFWLDVSHNRLEALPNSLLAPLGRLRYLSLRNNSLRTFTPQPPGLERLWLEGNPWDCGCPLKALRDFALQNPSAVPRFVQAICEGDDCQPPAYTYNNITCASPPEVVGLDLRDLSEAHFAPC